SEQUENCE LISTING (1 sequence)

SEQ ID NO: 1 - DNA, 1413 bp, Mouse cathepsin L gene
cggcagacttcttgtgcgcacgtagccgcctcaggtgtttgaaccggctttttaggattggtctaatcagatcctcatttttgttcccttcctaggttttaaaacatgaatcttttactccttttggctgtcctctgcttgggaacagccttagctactccaaaatttgatcaaacctttagtgcagagtggcaccagtggaagtccacgcacagaagactgtatggcacgaatgaggaagagtggaggagagcgatatgggagaagaacatgagaatcatccagctacacaacggggaatacagcaacgggcagcacggcttttccatggagatgaacgcctttggtgacatgaccaatgaggaattcaggcaggtggtgaatggctatcgccaccagaagcacaagaaggggaggctttttcaggaaccgctgatgcttaagatccccaagtctgtggactggagagaaaagggttgtgtgactcctgtgaagaaccagggccagtgcgggtcttgttgggcgtttagcgcatcgggttgcctagaaggacagatgttccttaagaccggcaaactgatctcactgagtgaacagaaccttgtggactgttctcacgctcaaggcaatcagggctgtaacggaggcctgatggattttgctttccagtacattaaggaaaatggaggtctggactcggaggagtcttacccctatgaagcaaaggacggatcttgtaaatacagagccgagttcgctgtggctaatgacacagggttcgtggatatccctcagcaagagaaagccctcatgaaggctgtggcgactgtggggcctatttctgttgctatggacgcaagccatccgtctctccagttctatagttcaggcatctactatgaacccaactgtagcagcaagaacctcgaccatggggttctgttggtgggctatggctatgaaggaacagattcaaataagaataaatattggcttgtcaagaacagctggggaagtgaatggggtatggaaggctacatcaaaatagccaaagaccgggacaaccactgtggacttgccaccgcggccagctatcctgtcgtgaattgatgggtagcggtaatgaggacttatggacactatgtccaaaggaattcagcttaaaactgaccaaacccttattgagtcaaaccatggtacttgaatcattgaggatccaagtcatgatttgaattctgttgccatttttacatgggttaaatgttaccactacttaaaactcctgttataaacagctttataatattgaaaacttagtgcttaattctgagtctggaatatttgttttatataaaggttgtataaaactttctttacctcttaaaaataaattttagctcagtgtgtgtgtcg

(19) United States
(12) Patent Application Publication
(10) Pub. No.: US 2003/0031658 A1
(43) Pub. Date: Feb. 13, 2003

(54) Targeting of endosomal growth factor processing as anti-cancer therapy

(76) Inventors: Pnina Brodt, Montreal (CA); Roya Navab, Toronto (CA)

Correspondence Address: KLAUBER & JACKSON, 411 HACKENSACK AVENUE, HACKENSACK, NJ, 07601

(21) Appl. No.: 10/170,219

(22) Filed: Jun. 12, 2002

Related U.S. Application Data
(63) Continuation of application No. PCT/CA00/01460, filed on Dec. 6, 2000.
(60) Provisional application No. 60/170,777, filed on Dec. 15, 1999.
(60) Provisional application No. 60/233,484, filed on Sep. 19, 2000.

(51) Int. Cl. A61K 48/00

(52) U.S. Cl. 424/93.21; 514/44

(57) ABSTRACT

The present invention relates to targeting of growth factor processing for the prevention of tumor cell proliferation and/or for the induction of tumor cell apoptosis or the spontaneously “collapsing” (suicidal) tumors and therapeutical methods thereof. More precisely, the present invention relates to an anti-cancer compound for preventing tumor cell proliferation and/or inducing tumor cell apoptosis, which comprises a compound specifically targeted directly or indirectly at an endosomal enzyme involved in cellular processing of a growth factor, regulation of growth factor mediated signaling growth factor receptor turnover and tumorigenicity.

### BACKGROUND OF THE INVENTION

(a) Field of the Invention

The invention relates to targeting of growth factors processing for the prevention of tumor cell proliferation and/or for the induction of tumor cell apoptosis or the spontaneously “collapsing” (suicidal) tumors and therapeutical methods thereof.

(b) Description of Prior Art

Apoptosis-programmed cell death is a complex process whose centrality to normal development and the maintenance of tissue homeostasis have become increasingly clear in recent years. Cancer cells often acquire resistance to apoptotic signals through deregulated expression of oncogens and suppressor genes and/or through altered growth factor and growth factor receptor expression. This escape from apoptosis contributes to the problematic resistance of cancer cells to conventional cancer therapy.

The ability of the cancerous cells to invade adjacent tissue and disseminate to distant sites or to metastasize, is the primary cause of death for most patients with cancer. The past thirty years have seen dramatic increases in our understanding of the metastatic process. Research has demonstrated that metastasis is not a random process but rather a series of sequential steps, the individual outcome of which depends on the interactions of the cancer cells with their microenvironment (Fidler, 1990). The steps in the metastatic process are interrelated and failure at any one of these stages aborts the process (Fidler, 1990). Recent advances have led to identification of molecular mediators and mechanisms underlying the process of metastasis. These include isolation and characterization of families of molecules involved in regulation of angiogenesis, cell-cell and cell-matrix adhesion, proteolysis, migration and growth. This improved understanding of the complex process of cancer progression has been the impetus for a recent worldwide effort to develop new diagnostic tools and therapeutic reagents targeting molecular mediators of metastases.

One step crucial for invasion and metastasis is the proteolytic degradation of the extracellular matrix (ECM) (Liotta et al., 1986). Among several families of proteolytic enzymes implicated in this degradative process, are the lysosomal cysteine proteinases cathepsin B and L (Sloane, 1990).

In the past decade, inhibitors of the cathepsins, in particular, cathepsins B, L and D have been developed as potential anti-metastatic agents. Human tumors generally express higher levels of these enzymes than normal tissues. As evidence continues to accumulate on factors distinguishing highly metastatic cells from those with lower or non-invasive properties, it has become clear that the more invasive cell types have both increased cysteine proteinase activity and decreased levels of endogenous cysteine protease inhibitors (Lumkowski et al., 1997). These proteinases may contribute to invasion directly through extracellular matrix degradation but also indirectly by controlling the turnover of growth factor receptors involved in regulation of proteinase gene expression.

Tumor H-59 is a highly metastatic variant of the Lewis lung carcinoma which produces high levels of cathepsin L and MMP-2 but low levels of cathepsin B (Brodt et al., 1992). Previously, we have shown that E-64, a natural specific inhibitor of cysteine proteinases inhibited liver colonization by these tumor cells, whereas PRCB1 a specific inhibitor of cathepsin B (Navab et al., 1997) had no effect. In addition, to their role in invasion, evidence has recently emerged that the cysteine proteinases play a role in regulation of cell survival and growth (Xing et al., 1998).

It would be highly desirable to be provided with the targeting of growth factors processing for the prevention of tumor cell proliferation and/or for the inhibition of tumor metastases through spontaneous induction of tumor cell apoptosis resulting in “collapsing” (suicidal) tumors.

### SUMMARY OF THE INVENTION

One aim of the present invention is to provide the targeting of growth factors processing for the prevention of tumor cell proliferation and/or for the induction of tumor cell apoptosis leading to spontaneously “collapsing” (suicidal) tumors.

In accordance with the present invention there is provided an anti-cancer compound for preventing tumor cell proliferation and/or inducing tumor cell apoptosis, which comprises a compound specifically targeted directly or indirectly at an endosomal enzyme involved in cellular processing of a growth factor, regulation of growth factor mediated signaling growth factor receptor turnover and tumorigenicity.

The preferred anti-cancer compound in accordance is selected from the group consisting of chemical compounds comprising E-64, CA-074 and analogues thereof and antisense of cathepsins B, H, L, and S.

Preferably, the growth factor is selected from the group consisting of IGF-I, IGF-II, TGFα, PDGF, EGF, HGF, FGF, VEGF and others acting via tyrosine kinase receptors.

Preferably, the anti-cancer compound in accordance with the present invention is an antisense which comprises a mRNA sequence capable of hybridizing to a cathepsin mRNA selected form the group consisting of sequence complementary to a cathepsin mRNA and fragments thereof. More preferably, such an antisense comprises a sequence selected from the group consisting of a 300 bp fragment spanning nucleotides 511-810 of mouse cathepsin L gene set forth in SEQ. ID. NO. 1 or functional equivalent fragment thereof of homologue cathepsin L gene.

In accordance with the present invention there is also provided a method for the treatment of cancer in a patient, which comprises administering to the patient a therapeutically effective amount of an agent for inhibition of a endosomal proteinase expression, whereby inhibition of a proteinase expression causes an inhibition of growth factor degradation and cancer cell death.

Preferably, the proteinase is selected from the group consisting of endosomal cathepsins such as cathepsin B, H, L, and S.

Preferably and in accordance with the present invention, the cancer cells are metastases.

In accordance with the present invention there is provided a method of screening for compounds with anti-cancer activity, which comprises the steps of:

a) treating a cell line dependent on a growth factor receptor where a cathepsin is involved in its turn over with a compound; and

b) determining viability of the cell line, wherein apoptosis of said cell line is indicative of a compound having anti-cancer activity.

The anti-cancer activity is preferably an anti-metastatic activity.

Preferably, the cathepsin is selected from the group consisting of cathepsin B, H, L and S.

In accordance with the present invention there is also provided the use of a cell line for screening compounds with anti-cancer activity, wherein the cell line is dependent on a growth factor receptor where a cathepsin is involved in its turn over.

The use of the cell line in accordance with a preferred embodiment of the present invention, wherein the cell line is tumor H-59.

The use of the cell line in accordance with a preferred embodiment of the present invention, wherein the cathepsin is selected from the group consisting of cathepsin B, H, L and S.

In accordance with the present invention there is provided an anti-cancer compound, which comprises a compound blocking intracellular growth factor degradation whereby growth factor-induced cellular proliferation is inhibited.

### BRIEF DESCRIPTION OF THE DRAWINGS

FIG. 1 illustrates reduced expression of cathepsin L in antisense transfected clone (CLAS-1);

FIG. 2 illustrates Western blot analysis of cathepsin L synthesis in the antisense transfected cells;

FIG. 3 illustrates inhibition of H-59 invasion by antisense cathepsin L transfectant cells;

FIG. 4 illustrates reduction in the cloning efficiency of antisense transfected clone;

FIG. 5 illustrates inhibition of the proliferative response to IGF-1 in cathepsin L antisense transfected cells;

FIG. 6 illustrates Zymographic analysis of MMP-2 activity in cathepsin L antisense transfected clone;

FIG. 7 illustrates inhibition of liver colonization by cathepsin L antisense transfected cells; and

FIG. 8 illustrates the DNA sequence of mouse cathepsin L gene (SEQ ID NO:1).

FIG. 9 illustrates the Loss of IGF-IR functions in tumor cells treated with the cysteine proteinase inhibitor E-64.

FIG. 10 illustrates the cysteine proteinase inhibitor E-64 blocking endosomal IGF-I degradation.

FIG. 11 illustrates that E-64 treatment causes a reduction in post-ligand binding cell surface receptor expression without affecting IGF-IR synthesis.

FIG. 12 illustrates the increased levels of tyrosine phosphorylated IGF-I, receptor in E-64 treated tumor cells.

FIG. 13 illustrates that inhibition of cysteine proteinase activity leads to alteration of IGF-IR signal transduction.

### DETAILED DESCRIPTION OF THE INVENTION

In accordance with the present invention, there is demonstrated for the first time that the suppression of synthesis of an endosomal cysteine proteinase can lead to a reduction in growth factor binding sites and to a loss in the ability to respond to growth factor, which causes the tumor to loose its ability to proliferate and invade.

Furthermore, there is demonstrated that the suppression of the target that act in the regulation of cell survival and growth is in connection with the spontaneous cell death.

The present invention also demonstrated that chemicals and/or antisenses which block the activity or synthesis of the enzymes which process the growth factors inhibit tumor proliferation.

The present invention will be more readily understood by referring to the following examples which are given to illustrate the invention rather than to limit its scope.

### EXAMPLE I

### Antisense for Induction of Tumor Cell Apoptosis

Several lysosomal proteinases including the cysteine proteinase cathepsin L, have been implicated in malignant progression of tumors. Many investigators have demonstrated correlations between increased activity of cathepsin L and increased metastatic capability of animal tumors or malignancy of human tumors. Here, the role of cathepsin L in metastasis was further investigated using H-59 cells transfected with a plasmid vector expressing CL cDNA in the antisense orientation. Among the transfectant clones, a few and mostly one clone (CLAS-1) showed reduction in both mRNA expression and synthesis of cathepsin L. These cells had markedly reduced invasion in a reconstituted basement membrane (98%) as compared with that of controls. These cells had a significant decrease in MMP-2 synthesis as assessed by gelatin zymography. The CLAS-1 cells had a reduction in IGF-1 binding sites and lost the ability to respond to IGF-1. When injected in vivo, directly into the microvasculature of the liver (experimental metastasis), these cells had reduced numbers of metastases under conditions which allowed wild-type or control transfectants to form multiple hepatic metastases. The results demonstrate that cathepsin L can play a critical role in the regulation of carcinoma metastasis.

Materials and Methods

Cell Lines

Tumor H-59 was established from a hepatic metastases of the parent line 3LL (Brodt, 1986). The tumor was maintained in vivo by s.c. implantation of liver metastases derived from tumor-bearing mice into new recipient animals. In vitro monolayer cultures of the tumor were maintained in RPMI containing 10% FCS as detailed elsewhere (Brodt, 1992).

Construction of Cathepsin L Plasmids

An XbaI-EcoRI fragment corresponding to the first 300 base pairs of the cathepsin L cDNA was ligated into the EcoRI-XbaI site of the PSVK3 plasmid vector (Pharmacia) in the antisense orientation relative to the SV40 early promoter gene. This Plasmid also expresses a neomycin resistance (Neo^{R}) gene under the control of an SV40 promoter that confers resistance to Neomycin. Cloning of the cathepsin L cDNA in the antisense orientation was confirmed by restriction analysis.

Transfections

The plasmid designed to produce antisense cathepsin L, was introduced into H-59 cells by coprecipitation with calcium phosphate and the cells cultured in RPMI 1640 containing 10% FCS, which was supplemented from day 2 onward with 100 μg/ml G-418 (GIBCO-BRL, Burlington, Ontario, Canada). Stable G418-resistant transformants were isolated 12-14 days later.

Northern Blot Analysis

Cellular RNA was extracted from H-59 and transfected cells by Trizol. A ³²P-labeled 1.19-Kb mouse cathepsin L cDNA fragment (a kind gift from Dr. Ann F. Chambers, London Regional Cancer Center, London, Ontario, Canada) and an 800-bp fragment of rat cyclophilin cDNA were used as hybridization probes. The relative amounts of mRNA transcripts were analyzed by laser densitometry using an Ultroscan XL enhanced laser densitometer and normalized relative to the internal cyclophilin controls.

Western Blot Analysis

Western blot analysis was essentially as described previously (Brodt, 1992). Briefly, serum-free conditioned media (60×concentrated) from transfected and non-transfected H-59 tumor cells, were separated on a 12.5% SDS-polyacrylamide gel and the proteins electrophoretically transferred onto nitocellulose filters (0.2 mm). The blots were probed with a rabbit antiserum to human recombinant procathepsin L at a dilution of 1:100. As a standard, human cathepsin L was run in a separate lane (1 μg/μl). Alkaline phosphatase-conjugated affinity purified goat anti-rabbit IgG (Bio/Can Scientific, Mississauga, Ontario) was used as a second antibody at a dilution of 1:1000.

Gelatin Zymography

The gelatinolytic activity of MMP-2 was analyzed by zymography as described previously (Brodt et al., 1992). The concentrated conditioned media (×60) from transfected and non-transfected clones which were cultured for 48 h and were electrophoresed on a 10% SDS-polyacrylamide gel containing 1 mg/ml gelatin. The gels were stained with Coomassie Blue and destained with 10% acetic acid-50% methanol until the desired color intensity was obtained. The gelatinolytic activity seen as a clear zone on the blue background was quantitated by densitometry using photographic negatives of the gel.

Soft Agar Cloning Assay

To measure anchorage-independent growth, a soft agar cloning assay was used. Briefly, tumor cells, transfected and non-transfected, were mixed with a solution of 0.8% agar (Difco Laboratories Inc., Detroit, Mich.) added to an equal volume of a 2×concentrated RPMI-FCS medium and plated in six-well plates (Fisher Scientific, Montreal, Quebec) on solidified 2% agar at a concentration of 10⁴ cells/well. The overlay was allowed to solidify and then supplemented with 1 ml RPMI-FCS containing G418. The medium was replenished on alternate days for 12 days. Colonies were enumerated using an inverted microscope (Diaphot-TMD Inverted, Nikon Canada).

Tumor Cell Proliferation Assay

H-59 cells and transfectants were cultured in SF-RPMI for 24-h and then dispersed and seeded into 96-well plates (Falcon, Lincoln Park, N.J.) at a density of 2×10³ cells/well and incubated for 54 h with medium containing IGF-I as we described previously (Long et al., 1994). The cells were pulsed with 0.1 mCi/ml of [³H] thymidine (Du Pont Canada, Mississauga, Ontario, Canada) for 18 h, and thymidine incorporation was monitored as detailed elsewhere (Long et al., 1995).

Ligand-Binding Assay

IGF-1 binding sites were quantitated as we previously described (Long et al., 1994). Briefly, transfected and non-transfected H-59 cells were cultured with RPMI-FCS containing G418 in 24-well plates for 2-3 days. The culture medium was removed and replaced with fresh medium. The binding assay was carried out 24 h later. To each well, 8-1500 pM of ¹²⁵I-labeled IGF-1 in binding medium (SF-RPMI containing 1 mg/ml BSA and 1 μg/ml leupeptin) were added, with or without graded concentrations of unlabeled IGF-1 for a 1 h incubation at 37° C. The cells were rinsed twice with ice-cold binding medium and solubilized in 0.01 N NaOH containing 0.1% Triton™ X-100 and 0.1% SDS. The number of cells/well at the time of the assay was determined from triplicate control wells which were manipulated in the same manner. An aliquot was removed from each well and the radioactivity was measured in an LKB gamma counter. The number of IGF-1 binding sites were calculated using the Ligand program (Long et al., 1994).

Cell Invasion Assay

Tumor cell invasion was determined in vitro by the reconstituted basement membrane (Matrigel) invasion assay, essentially as described previously (Navab et al., 1997). Briefly, 60 μl of Matrigel (Collaborative Research, Bedford, Mass., USA) diluted to a concentration of 0.23 mg/ml were applied to 8 μm filters. These filters were dried overnight, reconstituted with serum-free RPMI and placed in 24-well plates. To each filter 5×10⁴ cells in 100 μl of RPMI medium containing 0.2% BSA were added. Rat fibronectin (5 μg/ml; Gibco BRL) was used as a chemoattractant in the lower chamber. Following a 48-h incubation at 37° C., the cells on the upper surface of the filter were removed with a cotton swab and the filters fixed in 0.1% glutaraldehyde and stained with 0.2% crystal violet. For each filter 20 random fields were counted using a Nikon inverted microscope (×100) and duplicate samples were analyzed for each assay condition. In each experiment, control filters were coated with 7.5 μg/filter of human placental type IV collagen (Sigma) to control for changes in cell migration.

Tunnel Assay

Apoptotic cells were detected by direct immunoproxidase detection of degoxigenin-labaled genomic DNA in thin sections of fixed tissue using the Apop Tag in situ apoptosis detection kit. Liver obtained from animals that were injected with 2×10⁵ transfected and non-transfected H-59 cells by the intrasplenic/portal route (i.s.) were fixed in 10% neutral buffered formalin followed by ethanol: acetic acid and embedded in paraffin. Sections were prepared, quenched in 2% hydrogen peroxide in PBS at room temperature and incubated with terminal deoxynucleotidyl transferase (TdT) for 1 hr at 37° C. Following this anti-digoxigenin -peroxidase was applied to the slides and after several washes in PBS color was developed using hydrogen peroxide and DAB (Diaminobenzidine) as substrates. The slides were counterstained with methyl green, dehydrated and mounted.

Liver Colonization Assay

Animals were injected with 2×10⁵ transfected and non-transfected H-59 cells by the intrasplenic/portal route (i.s.) and then immediately splenectomized as described previously (Long, 1995). The animals were sacrificed 14-21 days later, the livers removed and the metastases enumerated immediately.

Results and Discussion

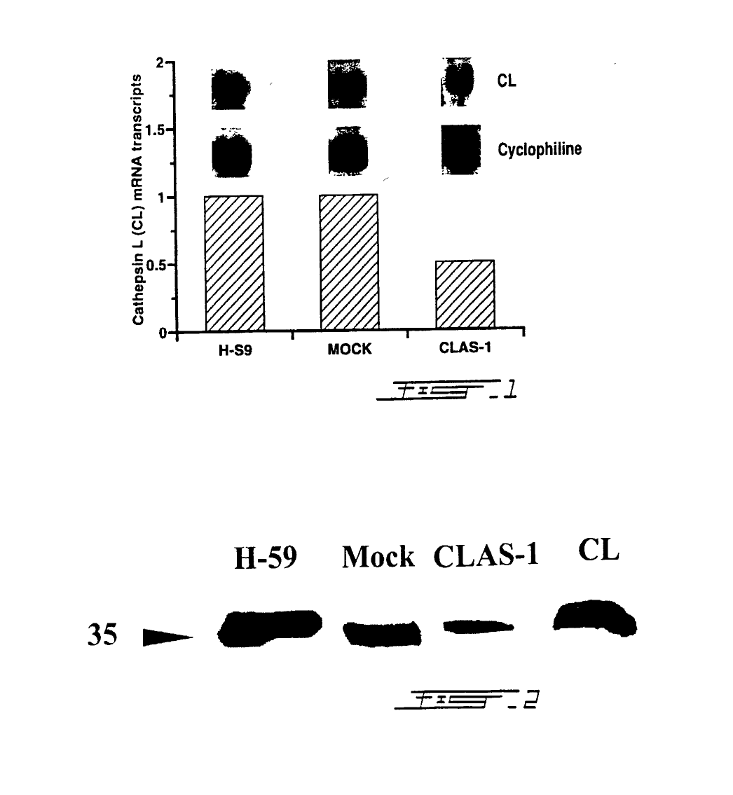

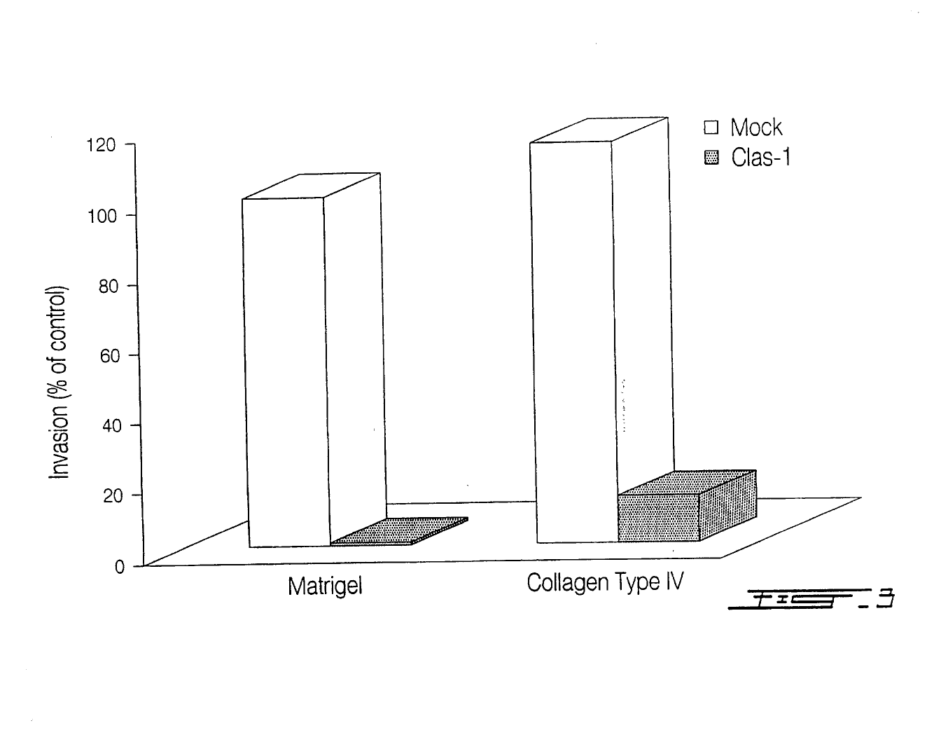

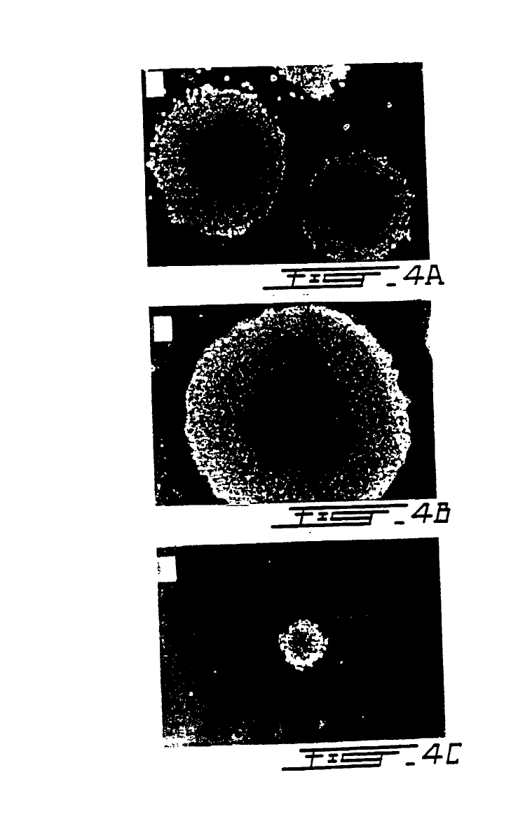

In accordance with the present invention we analyzed the role of cathepsin L in the invasion and metastasis of a highly invasive murine lung carcinoma subline H-59 cells, in which the constitutive expression of cathepsin L was suppressed by stable transfection with a plasmid vector expressing a 300 bp antisense fragment of cathepsin L cDNA in the antisense orientation relative to the promoter. One clone (CLAS-1) was isolated in which cathepsin L mRNA expression was 50% reduced relative to non or mock-transfected cells (FIG. 1) with a corresponding loss in protein synthesis (FIG. 2).

Using Northern blot analysis, Thirty μg of total RNA were loaded per lane. Blots were probed consecutively with ³²P-labeled 1.19-kb mouse cathepsin L cDNA and 800-bp rat cyclophiline cDNA fragments (FIG. 1). The intensity of the bands was measured by laser densitometry and is expressed as a ratio relative to the intensity of the cyclophiline bands.

Conditioned media derived from wild-type H-59, Mock transfected clone and antisense transfected clone (CLAS-1), were concentrated (60×) and the proteins (60 μg per lane) resolved on 12.5% SDS-polyacrylamide gel and transferred to a nitrocellulose filter. The filters were probed with a rabbit antiserum to human recombinant procathepsin L and normal human cathepsin L (CL) was used (1 μg/ml) as a control. The position of the procathepsin L is indicated with an arrow on the left (FIG. 2).

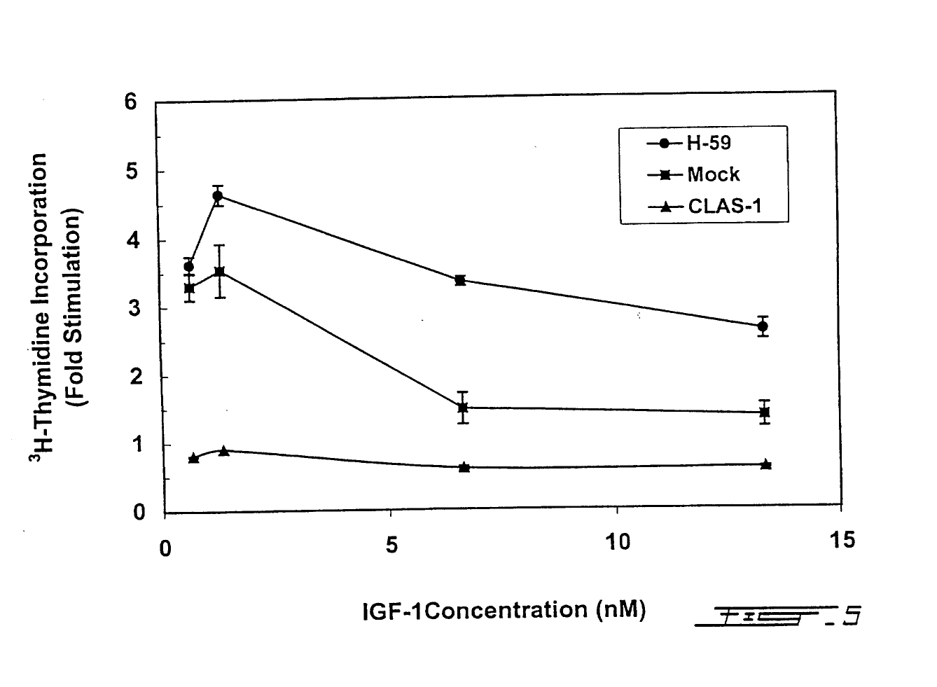

These cells had a significantly reduced invasion (99%) as measured in the reconstituted basement membrane (Matrigel) model (FIG. 3), as well as a significantly reduced (87%) migration on uncoated or 7.5 μg type IV collagen coated filters. Transfected and non-transfected H-59 cells (5×10⁴) were plated on Matrigel-coated filters and incubated for 48 h at 37° C. In each of the experiments, control filters were coated with human placental type IV collagen to control for changes in cell migration. Results are based on four experiments carried out in duplicate and are presented as percentage of invasion relative to control non-transfected cells.

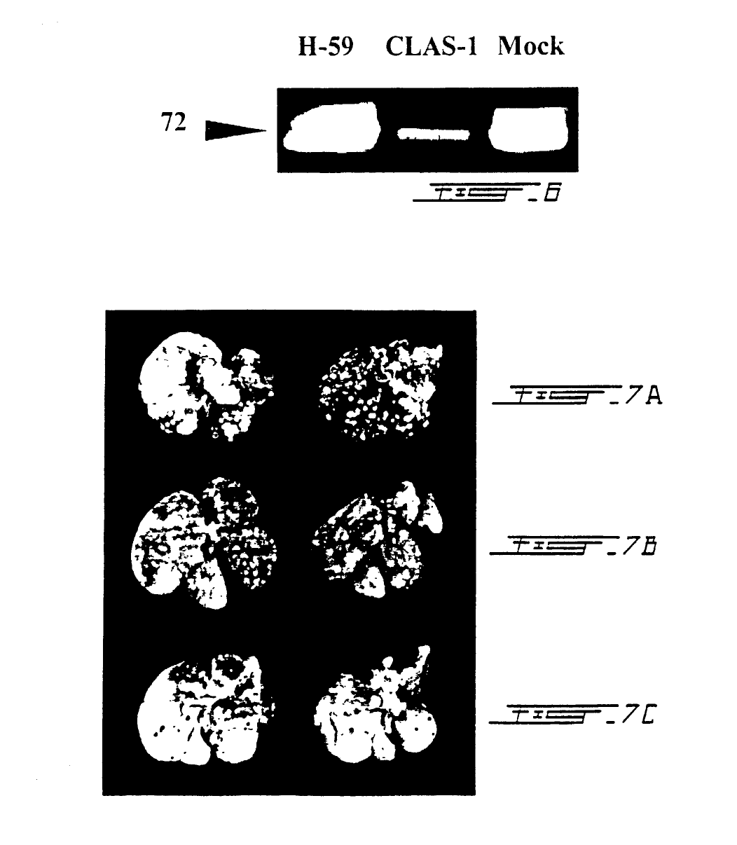

When the clonogenicity of these cells was measured in semi solid agarose, we found an 82% reduction in their cloning efficiency relative to control cells (Table 1, FIG. 4). Light microscopic view of the agar colonies from Table 1. Representative fields of control (a,b) and antisense transfected (c) cells (×250) are depicted in FIG. 4.

TABLE 1
Cathepsin L antisense transfected H-59 cells have a
reduced cloning efficiency in semi-solid agar

| | Number of colonies |
|---|---|
| H-59 | 287 ± 16.97 |
| Mock | 266.7 ± 42.67 |
| CLAS-1 | 52.5 ± 13.44 |

H-59 cells and antisense transfected cells were cultured in semi-solid agar for 12 days. Colonies which exceeded 250 μm in diameter were enumerated using a microscope equipped with an ocular grid. Results represent total number of colonies/plate and are expressed as means and SD of three plates per cell type.

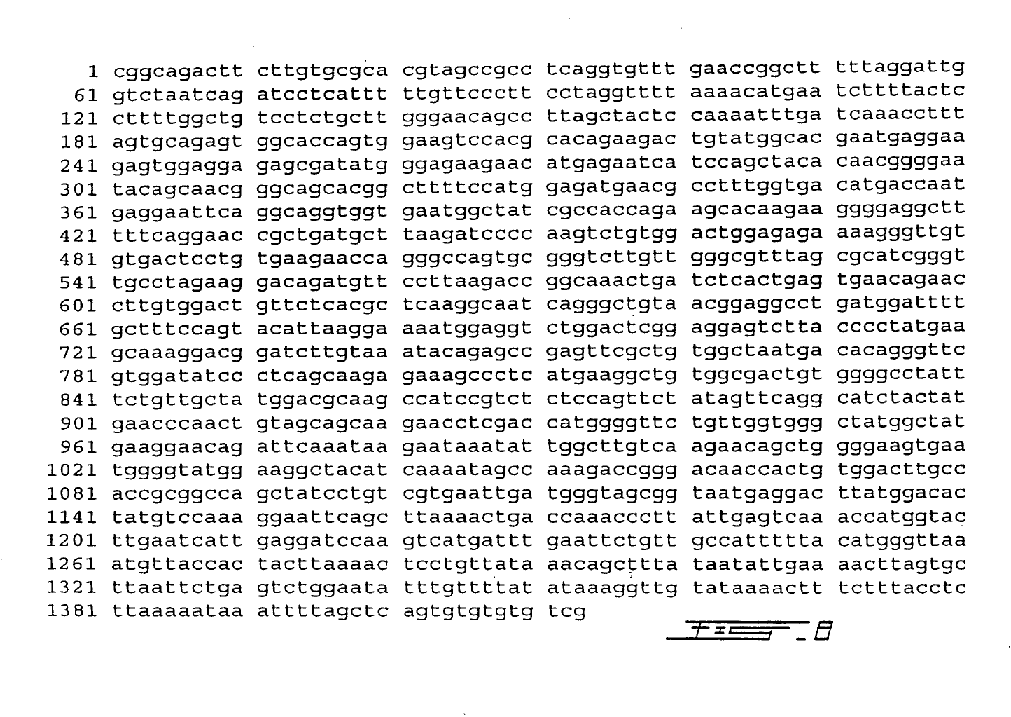

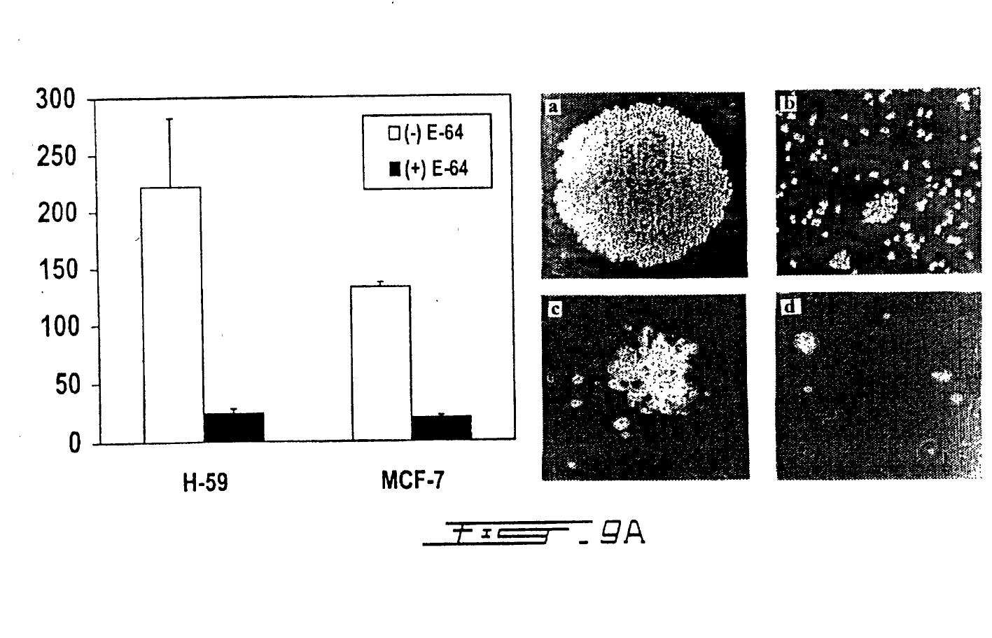

In monolayer cultures these cells lost their proliferative response to IGF-I (FIG. 5) associated with a 56-66% reduction in the number of IGF-I binding sites compared to controls as assessed by the ligand binding assay (Table 2). H-59 and transfected cells were seeded in 96-well microtiter plates in serum free medium and then incubated for 72 h with or without the indicated concentrations of IGF-1. The results represent means and SD of three experiments and are expressed as the increase in [³H] thymidine incorporation relative to cells incubated without IGF-1.

TABLE 2
Reduction of IGF-1 binding sites in cathepsin L
antisense transfectants clone (CLAS-1)

| | Binding site/cell |
|---|---|
| H-59 | 5.1 × 10⁵ |
| Mock | 3.96 × 10⁵ |
| CLAS-1 | 1.75 × 10⁵ |

Non-transfected and transfected H-59 cells were cultured in 24-well plates. To each well ¹²⁵I-IGF-1 was added at concentrations ranging from 8-1500 PM for 1 h incubation at 37° C. Triplicate wells were used for each concentration. Number of IGF-1 binding site/cell calculated from the means using the ligand program.

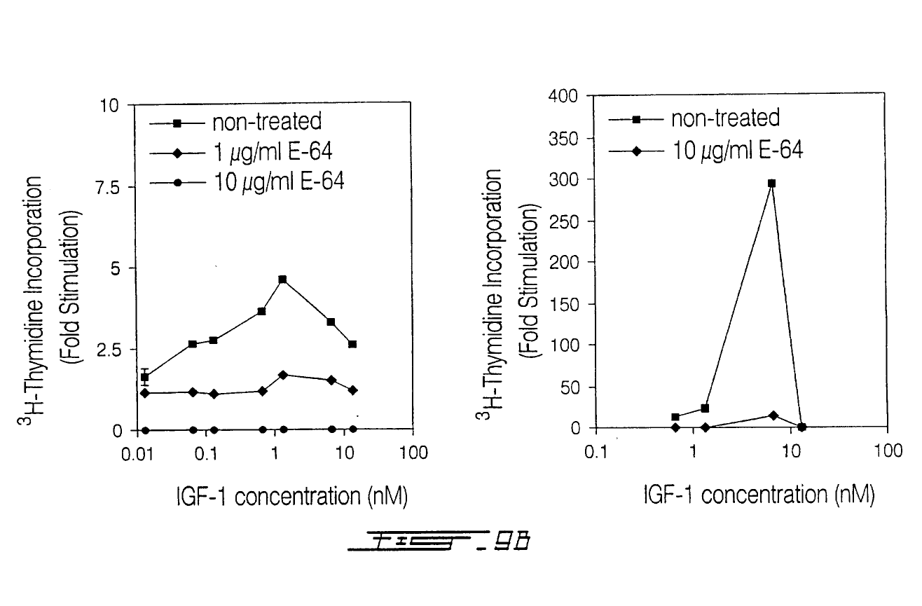

When the function of MMP-2 was investigated in antisense transfected CLAS-1 cells, we found a significant decrease in the level of MMP-2 mediated gelatinolytic activity, as assessed by gelatin zymography (FIG. 6). Concentrated condition media (×60) were separated by electrophoresis on 10% polyacrylamide gels containing 1 mg/ml gelatin. Shown are results obtained with antisense transfected and control H-59 cells.

Taken together with our previous studies which identified IGF-1R as a regulator of anchorage-independent growth, cellular proliferation, MMP-2 synthesis and invasion (Long et al., 1998a,b), in these cells, the results implicate cathepsin L activity in the regulation of the IGF-1R/IGF-1 system cellular functions.

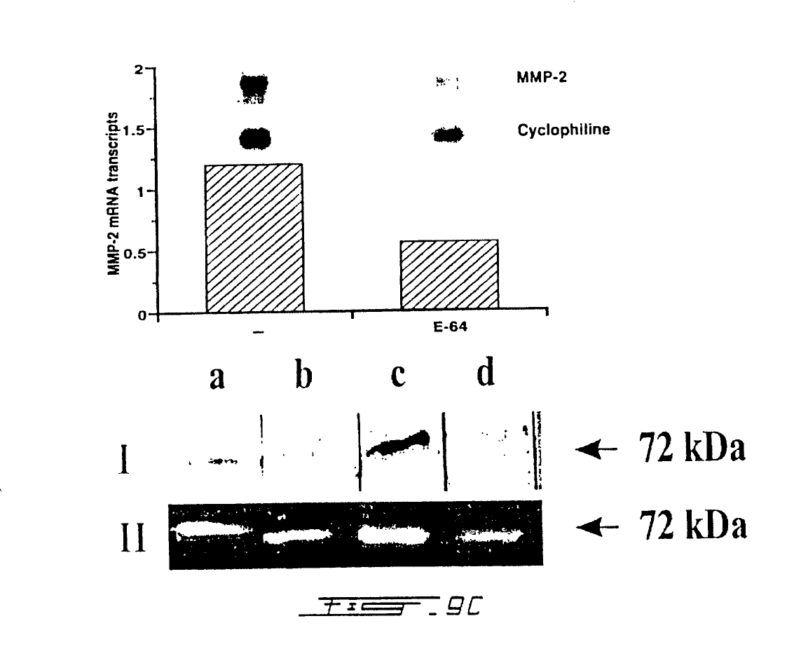

In vivo studies revealed that CLAS-1 cells had a significantly reduced ability (up to 70% reduction) to form hepatic metastasis following the intrasplenic/portal injection of 2×10⁵ cells, suggesting that cathepsin L is involved in regulation of liver colonization in this model (Table. 3, FIG. 7). Representative livers from Table 3 are shown. (A) non-transfected cells. (B) Control transfected cells (Mock). (C) Antisense cathepsin L transfected cells (CLAS-1).

TABLE 3
Cathepsin L antisense transfected H-59 carcinoma cells
block liver colonization

| | Median # of nodules |
|---|---|
| H-59 | 112.5 (36-147) |
| Mock | 149.5 (56-200) |
| CLAS-1 | 43.5 (29-84)* |

Experimental hepatic metastases were enumerated 14 days following i.s. inoculation of 2 × 10⁵ transfected H-59 cells. Results are based on 5-10 animals per each group.
*P = 0.004 relative to H-59 and P = 0.008 relative to Mock

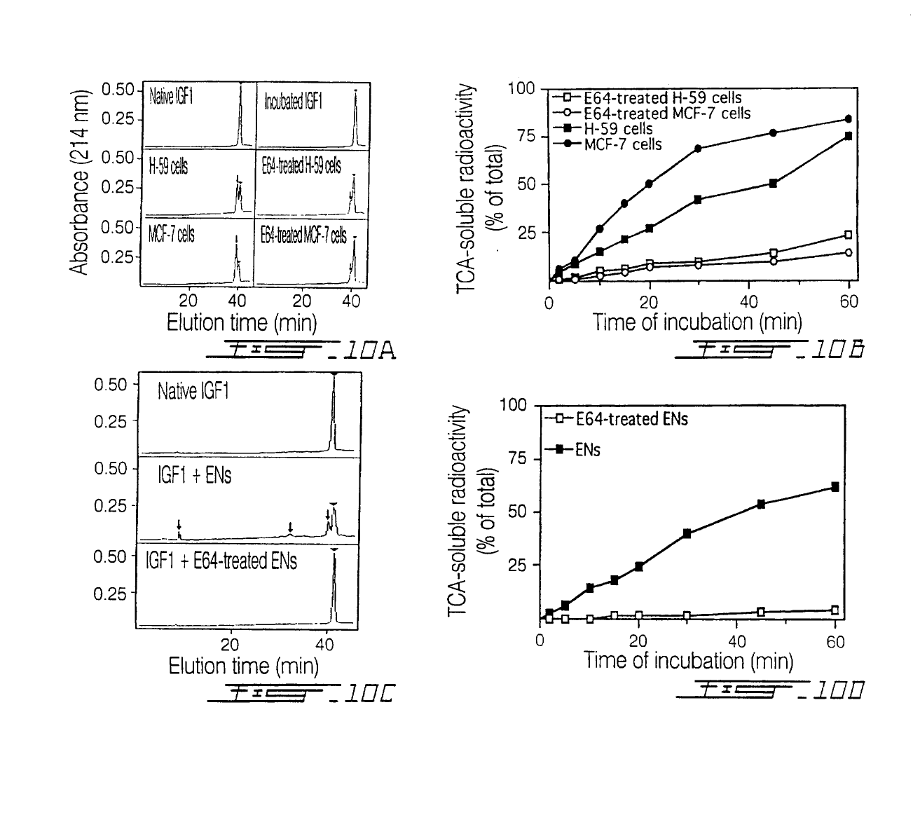

Interestingly, we observed in livers of CLAS-1—injected mice, small hemorrhagic lesions which were absent in liver of animals injected with mock-transfected cells and never observed in control H-59—injected animals (FIG. 7). Microscopic analyses of these lesions by the Tunnel assay revealed a high incidence of apoptotic cells which were absent in lesions of mice injected with control cells (FIG. 8A) This is the first report which directly implicates cathepsin L in liver metastases formation and in the regulation of cell survival.

An essential role for proteases in metastasis has long been suggested, but evidence from the literature for a role of a particular protease has often appeared confusing for several reasons. Most of the observations are correlative, often the conclusions are extrapolations from in vitro models, or conclusions are made from a variety of different tumors and cell lines among which comparisons are difficult. Direct in vivo evidence for a role of a particular protease in metastasis comes from only a few experiments in which specific inhibitors of the proteolytic activity are utilized or from in vivo molecular biology experiments in which a particular protease gene expression can be selectively increased or decreased. These types of in vivo experiments are difficult and have been successfully carried out in only a few examples. Our data are the first direct evidence for a role of cathepsin L in experimental liver metastasis. These results identify cathepsin L as a potential target for anti-metastatic therapy based on its role in the regulation of cell survival and growth.

This is the first known evidence for the involvement of the cysteine proteinase cathepsin L in regulation of growth factor receptor (IGF-1R) expression and function and for its role in promoting cell survival.

The spontaneous cell death seen in hepatic lesions is to our knowledge the first report of its kind and the first to be observed in connection with suppression of cathepsin L expression.

### EXAMPLE II

### Endosomal Processing of IGF-I as a Potential Target for Anti-cancer Therapy

Materials and Methods

Cell Lines and Tissues

H-59 is a highly metastatic subline of the Lewis lung carcinoma with metastatic predilection for the liver, (Brodt et al 1986). Human breast carcinoma cell line MCF-7 was a gift from Dr. Mader (Dept of Biochemistry, University of Montreal, PQ, Canada). Endosomal fractions were prepared from livers of male Sprague-Dawley rats after an 18 h period of fasting. The livers were homogenized and the endosomal fractions isolated by discontinuous sucrose gradient centrifugation and collected at the 0.25 M to 1.0 M sucrose interface (20, 21, 24). The soluble extract (ENs) from the endosomal fractions was isolated by freeze/thawing in 5 mM Na-phosphate pH 7.4, and disrupted in the same hypotonic medium using a small Dounce homogenizer (15 strokes with the tight Type A pestle) followed by centrifugation at 300,000×gav for 30 min as described previously (20, 21, 24).

Reagents and Antibodies

E-64 [trans-epoxysuccinyl-L-leucylamido (4-guanidino)-butane], Protein A-sepharose beads and MTT [3-(4,5-dimethylthiazol-2-yl)-2,5-diphenyltetrazolium bromide] (thiazolyl blue) were purchased from Sigma (St Louis, Mo.). CA074-methyl ester [N-(L-3trans-propylcarbamoyloxirane-2-carbonyl)-L-isoleucyl-L-proline] a pro-inhibitor of intracellular cathepsin B (25) was from Peptides International (Louisville, Ky., USA). [³H] thymidine (2.0 Ci/mmol) was from Du Pont Canada (Mississauga, Ontario, Canada). ¹²⁵I-labeled IGF-I (2000 Ci/mmol) used for the ligand binding assay was obtained from Amersham Canada (Oakville, Ontario, Canada). Human rIGF-1 used for the IGF-1 proteolysis assay was radioiodinated by the lactoperoxidase method as described previously for insulin (24) to specific activities of 350-500 Ci/mmol, and purified by gel filtration on Sephadex G-50. A 1.1-Kb type IV collagenase cDNA fragment was kindly provided by Dr. W. Stetler-Stevenson (NIH, Bethesda, Md.). A 700-bp IGF-IR cDNA fragment was a kind gift from Dr. M. Pollak (Lady Davis Research Institute, Montreal, PQ, Canada). The following antibodies were used: rabbit antiserum to MMP-2 (Ab-45), a kind gift from Dr. William Stetler-Stevenson (NIH), anti-phosphotyrosine mAb PT-66 from Sigma and RC20-H (Transduction Laboratories), mAb C-20 to the murine IGF-IR β subunit from Santa Cruz Biotechnology Inc. (Santa Cruz, Calif.), mAb αIR3 to human IGF-1R from Calbiochem (Cambridge, Mass.), horseradish peroxidase (HRP)-conjugated goat anti-mouse and goat anti-rabbit IgG antibodies from Bio-Rad (Mississauga, Ontario), alkaline phosphatase-conjugated affinity purified goat anti-rabbit IgG from Bio/Can Scientific, (Mississauga, ON).

Functional Assays for IGF-1R

Thymidine incorporation and soft agar cloning assays were performed as follows: Semi-confluent cultures of H-59 or MCF-7 were cultured in serum free-medium for 24 h with or without different concentrations of E-64, dispersed, seeded onto 96-well polystyrene plates (Falcon) and incubated with different concentrations of IGF-I and with or without E-64 for 54-h prior to pulsing with 0.1 mCi/ml of [³H] thymidine for 18 h. For soft agar cloning, the tumor cells were mixed with a solution of 0.8% agar added to an equal volume of a 2× concentrated RPMI-FCS medium with or without 10 μg/ml of E-64, plated on solidified 2% agar at a concentration of 10⁴ cells/plate and supplemented with 1 ml RPMI-FCS containing or not 10 μg/ml of E-64. This medium was replenished on alternate days for 12 days. IGF-1-mediated induction of MMP-2 synthesis was analyzed by Western blotting and by gelatin zymography performed as described (Long et al 1998 b) using concentrated (×60) serum-free media conditioned by H-59 cells for 48 hr in the presence or absence of IGF-1 and with or without 10 μg/ml E-64. Blots were probed with a 1:500 dilution of mAb Ab-45 to MMP-2 and an alkaline phosphatase conjugated affinity purified, goat anti rabbit IgG, diluted 1:2000. For Northern blotting, a ³²P-labeled 1.1-Kb human MMP-2 and an 800-bp rat cyclophilin cDNA fragment were used as hybridization probes.

Measurement of Cell Surface IGF-1 Receptors

The ligand-binding assay and fluorocytometry were used to measure cell surface IGF-1 receptors on the murine H-59 and human MCF-7 cells, respectively. Two day old H-59 cultures were replenished with fresh medium containing or not 10 μg/ml E-64 and the binding assay performed 24 h later using 8-1500 pM of ¹²⁵I-labeled IGF-1 with or without graded concentrations of unlabeled IGF-1. Incubation was for 1 h at 37° C. following which the cells were rinsed and lysed in 0.01 N NaOH containing 0.1% Triton X-100 and 0.1% SDS and the radioactivity measured. The number of cells/plate at the time of the assay was determined from triplicate control wells which were manipulated in a similar manner. The Ligand program (27, 28) was used to calculate the number of IGF-1 binding sites per cell. IGF-1 receptors on MCF-7 cells were immunofluorescence labeled using 5 μg/ml mAb αIR3 and an FITC-conjugated goat anti-mouse IgG (diluted 1:50). Prior to labeling, the cells were cultured for 24 h in SF-RPMI with or without 10 μg/ml E-64 then dispersed, reseeded at a density of 10⁵ cells/well into 96-well plates, stimulated with 10 ng/ml of IGF-1 for 10 min and incubated for an additional 30 min at 37° C. Labeled cells were fixed in PBS containing 1% formalin and analyzed using a FACS Calibur System (Becton-Dickinson, San Jose, Calif.).

Ligand Proteolysis Assays

Proteolysis of IGF-1 was measured using the soluble endosomal extract prepared from rat liver parenchyma (1 ng) and cell lysates (3-15 mg) derived from H-59 and MCF-7 cells cultured for 24 h with or without 10μg/ml E-64, lysed by incubation in 50 mM phosphate buffer pH 7.4 containing 0.5% Triton X-100, 0.5% deoxycholate and 0.2 M NaCl for 30 min at 4° C. and then clarified by centrifugation at 30000 g for 30 min. These preparations were incubated for various lengths of time at 37° C. with 10-⁶ M unlabeled or 50,000 cpm [¹²⁵I]-labeled IGF-1 in 200 or 400 μl of 50 mM citrate-phosphate pH 5, respectively. The integrity of the radiolabeled ligand was assessed by precipitation with 10% trichloroacetic acid (Authier et al, 1995). To measure proteolysis of the unlabeled IGF-1, the samples were acidified with acetic acid (15%) and immediately loaded onto a reverse-phase HPLC column. Reverse-phase HPLC was performed on a Waters model 600 liquid chromatograph equipped with a model U6K sample injector fitted with a 500 ml loop and a mBondapak C18 column (Waters, 0.39×30 cm, 10 mm particle size). Samples were chromatographed using as eluent a mixture of 0.1% TFA in water (solvent A) and 0.1% TFA in acetonitrile (solvent B) with a flow rate of 1 ml/min. Elution was carried out using two sequential linear gradients followed by an isocratic elution: an initial gradient of 0-20% solvent B (30 min) ; a second gradient of 20-39% solvent B (15 min); and a third isocratic elution of 39% solvent B (15 min). Eluates were monitored on-line for absorbance at 214 nm with a LC spectrophotometer.

Immunoprecipitation and Western Blot Analysis

MCF-7 and H-59 cells were treated with 10 ng of IGF-1 for 5 min following or not pre-treatment with E-64 or CA074-ME as described above. Cells were then washed with PBS, solubilized in 30 mM Hepes pH 7.4, 150 mM NaCl, 1% Triton X-100, and spun at maximal speed in a microfuge for 15 min. Cell lysates (1 to 3 mg) were then immunoprecipitated respectively with anti-Shc, anti-IRS-1 or anti-IGF-IR antibodies overnight at 4° C. Immunoprecipitates were collected by addition of Protein A-Sepharose beads, washed three times with lysis buffer and resuspended finally in Laemmli sample buffer (Long et al 1986a). Immunoprecipitates were resolved by SDS-PAGE and transferred onto nitrocellulose membranes followed by immunoblotting with anti-phosphotyrosine antibodies or with antibodies to IRS-1, Shc or IGF-1R followed by HRP—conjugated goat anti-mouse or goat anti-rabbit IgG antibodies. The blots were revealed by enhanced chemilluminescence followed by radioautography on X-OMAT AR films.

Results and Discussion

Abrogation of IGF-IR Functions by the Cysteine Proteinase Inhibitor E-64.

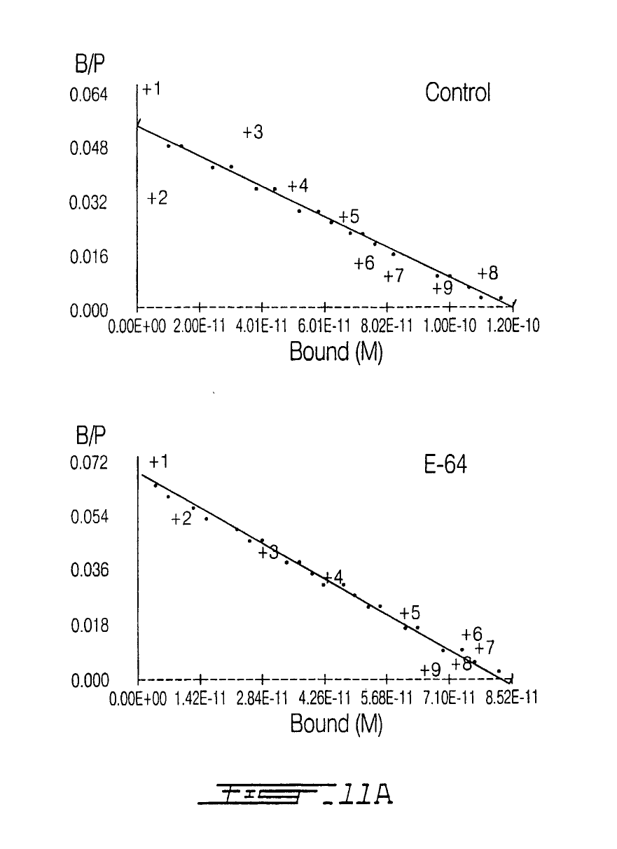

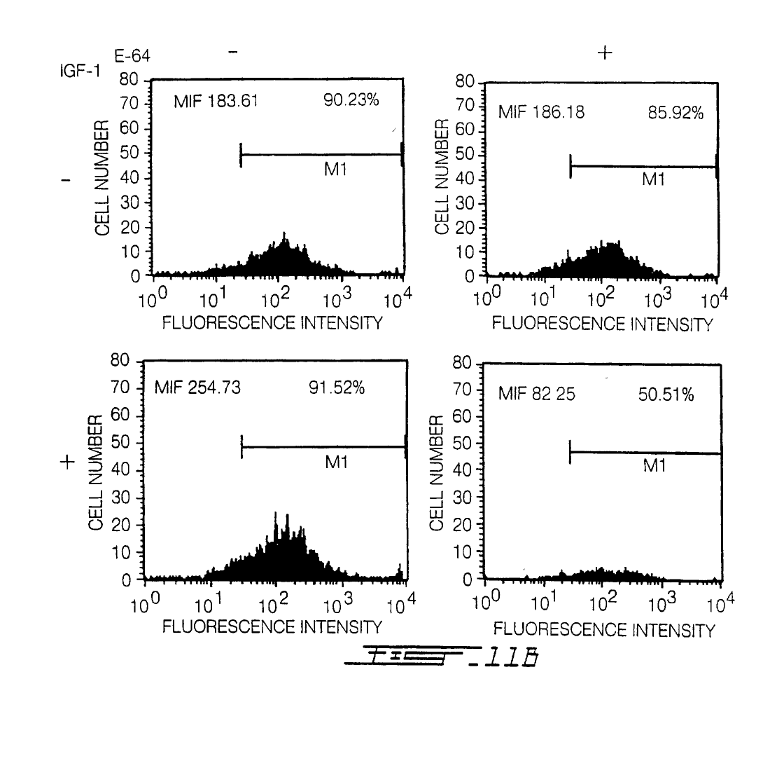

Cellular proliferation, anchorage independent growth and production of the matrix metalloproteinase MMP-2 are three IGF-I regulated cellular functions which are critical to the expression of the malignant phenotype. Treatment of MCF-7 and H-59 cells with the cysteine proteinase inhibitor E-64 at the non-toxic concentration of 10 μg/ml (Navab et al, 1997 ) abolished IGF-I induced proliferation and reduced by factors of 7 and 10 respectively, the cloning efficiency of these cells in semi-solid agar (FIG. 9A). The incorporation of ³H-thymidine in response to IGF-I was also completely abrogated in both cell lines (FIG. 9B). Furthermore, MMP-2 mRNA synthesis which is regulated by IGF-I (9) was reduced 2 fold. This was also reflected in decreased MMP-2 production and activity as determined by Western blotting and gelatin zymography, respectively (FIG. 9C).

E-64 Inhibits Endosomal Proteolysis of IGF-I

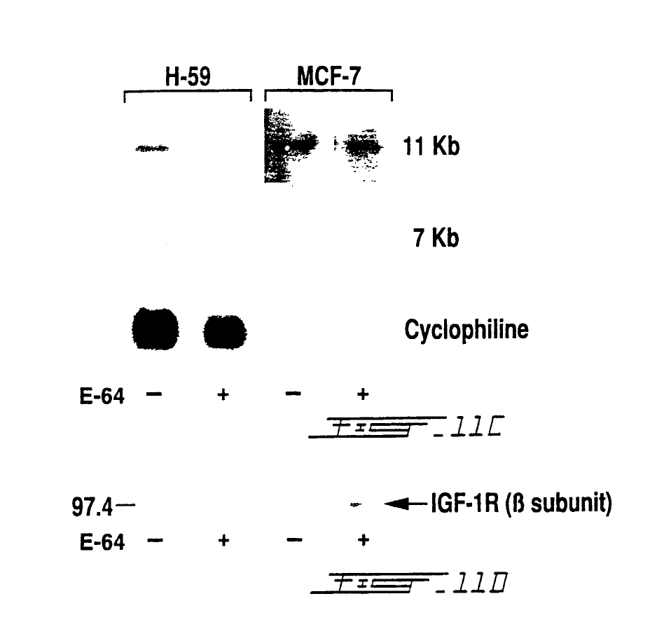

Endosomal endopeptidases such as Cathepsin B are inhibited by E-64 and have been implicated in the processing of receptor-ligand complexes (Authier et al 1995). We postulated that IGF-I receptor-mediated cellular functions in E-64-treated cells were blocked as a consequence of perturbed endosomal processing of the internalized receptor-bound IGF-I. Changes in IGF-I proteolysis were therefore investigated in lysates of E-64-treated tumor cells as well as in isolated liver parenchymal endosomal fractions which were incubated with exogenous IGF-I at acidic pH. Reverse phase HPLC analysis revealed that IGF-I degradation products which were detectable in the untreated preparations were absent following E-64 treatment (FIG. 10 A and C). This was subsequently confirmed when cell lysates and endosomal fractions were incubated with radioiodinated IGF-I for 1 hr and trichloroacetic acid (TCA) precipitation used to monitor ligand integrity. An increase in TCA-soluble radioactivity over time was evident in the untreated preparation but this was completely abolished by E-64 pretreatment (FIG. 10B and D) indicating that IGF-I proteolysis was blocked.

Reduced Cell Surface Levels of IGF-IR in E-64 Treated Cells

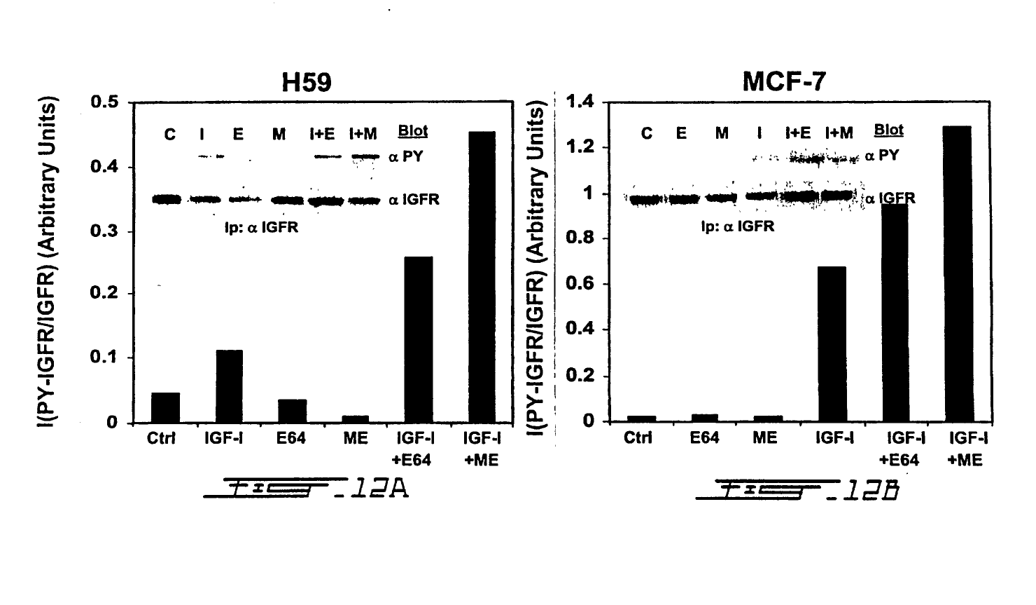

One possible consequence of ligand proteolysis blockade is the endosomal trapping of receptor-ligand complexes leading to a decreased availability of free receptor for recycling at the cell surface. We measured the effect of E-64 treatment on the levels of IGF-I receptor expression at the cell surface on H-59 and MCF-7 cells. Ligand-binding analysis revealed that the number of IGF-I binding sites measured after the addition of ¹²⁵I-IGF-I to H-59 cells was reduced by more than 2 fold, from 3.9×10⁵ sites/cell on untreated to 1.8×10⁵ sites/cell on E-64 treated cells (FIG. 11A) Flow cytometric analysis with a monoclonal antibody (mAb αIR3) to the a subunit of the human IGF-I receptor revealed that 40 min after the addition of ligand to serum starved MCF-7 cells, there was a reduction of 45% in the number of immunolabeled cells with the mean intensity of fluorescence declining from 255 to 82 (FIG. 11B). In neither of these cell types did E-64 treatment cause a reduction in IGF-IR mRNA levels (FIG. 11C) nor in the total level of immunoprecipitable receptor (FIG. 11D). These experiments suggested that the reduction of IGF-IR expression at the cell surface was not due to a change in receptor transcription or translation.

Increased Levels of Tyrosine Phosphorylated IGF-IR and Substrates in Cells Treated with Cysteine Proteinase Inhibitors

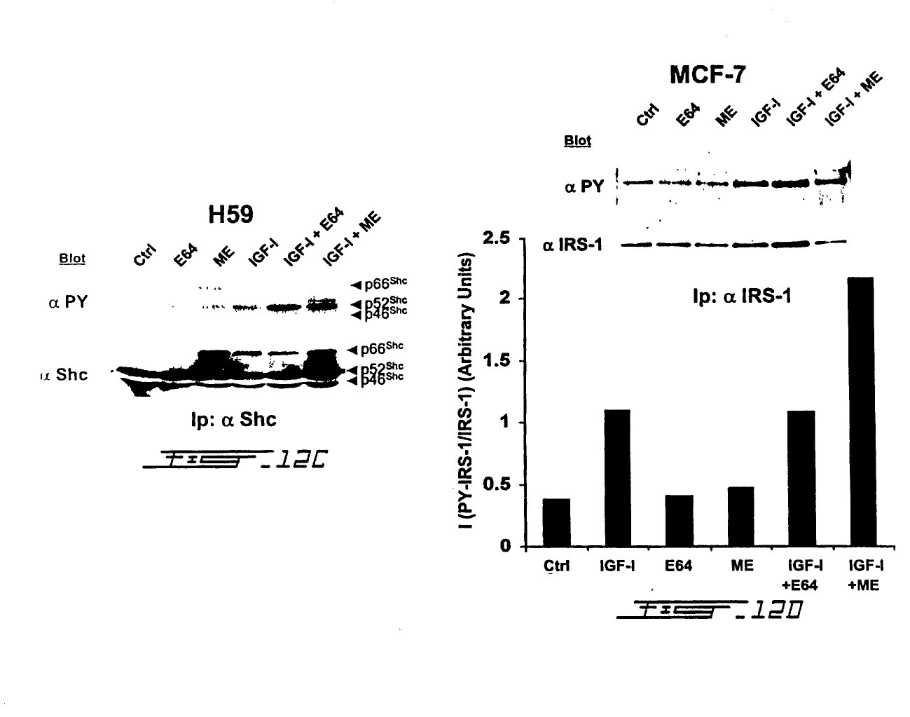

One of the earliest molecular events in IGF-IR ligand-induced signaling is the autophosphorylation of tyrosine residues on the receptor β subunit and the subsequent phosphorylation of downstream substrates such as IRS-1 and Shc. We first measured ligand induced tyrosine phosphorylation of the receptor in E-64 treated cells by immunoprecipitation with anti-IGF-IR antibodies followed by immunoblotting with anti-phospho-tyrosine antibodies. The total amount of tyrosine phosphorylated receptor β subunit in the inhibitor-treated cells increased by 2.5 fold relative to controls in H-59 cells and by 1.8 in MCF-7 cells (FIG. 12A and B). Moreover, in H-59 cells we also observed an increase in ligand-induced tyrosine phosphorylation of p52^{shc} while in MCF-7 cells an increase in tyrosine phosphorylated IRS-1 was noted (FIG. 12C & 12D). In these experiments we also tested the E-64 derivative, CA074-methyl ester-(CA074-ME), a specific pro-inhibitor for intracellular cathepsin B. Similarly to E-64, this inhibitor blocked cellular proliferation in response to IGF-I. In CA-074 ME treated cells, we also observed increased levels of immunoprecipitable, tyrosine phosphorylated receptor and substrates which generally exceeded those observed with E-64 (FIG. 12A-D).

Our results show that inhibition of cysteine proteinase activity by E-64 resulted in reduced cell surface IGF-IR expression levels and in the abrogation of cellular responses to IGF-I. In an apparent paradox however, treatment with this or a second cathepsin B inhibitor, CA074-ME also caused an increase in the levels of tyrosine phosphorylated IGF-IR β subunit, IRS-1 and Shc.

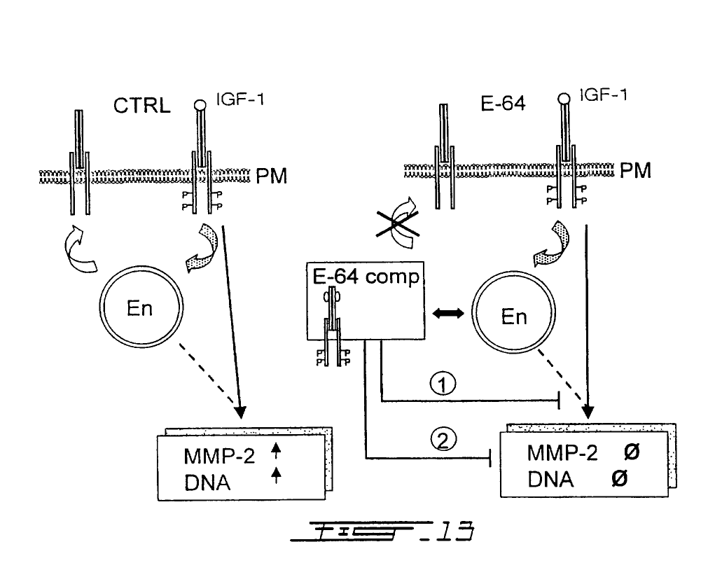

When taken together with our findings that IGF-I proteolysis was blocked in E-64-treated liver parenchymal endosomes and in tumor cell lysates, our results are consistent with a model whereby the inhibition of processing of the IGF-IR:IGF-I complex leads to “trapping” of the receptor-ligand complex in a subcellular compartment with two major consequences: (i) receptor recycling to the plasma membrane is dramatically decreased and (ii) IGF-IR β subunit and the IRS-1/Shc substrates remain hyperphosphorylated and this attenuates rather than activates IGF-IR mediated biological functions such as induction of DNA synthesis and MMP-2 transcription. We propose a model (FIG. 13) whereby the creation of an E-64 sensitive compartment that accumulates hyperphosphorylated IGF-IR either traps signaling molecules, preventing them from accessing normal signaling pathways in the cytoplasm (FIG. 13A) or activates new signaling pathways which inhibit DNA synthesis and MMP-2 mRNA transcription (FIG. 13B). Support for this model comes from other studies of receptor/ligand trafficking in different models. Receptor phosphorylation and signaling within the endosomal compartment has been demonstrated for the insulin receptor kinase and EGFR activation and Shc recrultment within endosomes have also been observed (Authier et al, 1999). Reports have linked receptor internalization to the activation of the Shc/MAPK pathway while mutant receptors which were accumulating in non-endosomal compartments presented impaired signaling pathways (Dews et al, 2000) due to differential sequestration of enzymes and substrates.

It has been clearly shown that ligand degradation is a key event in receptor recycling and signaling (Authier et al, 1999). Indeed, preventing degradation of insulin in endosomes using the H2 analogue led to a higher receptor concentration and tyrosine autophosphorylation of the receptor β subunit in this organelle. In this study endosomal proteolysis of the H2 analogue was also slowed as a result of an increased residence time of the analogue on the insulin receptor and a low affinity of endosomal acidic insulinase for the dissociated H2 molecule. The results we show here suggest that the underlying mechanisms in both systems were similar. Also relevant in this context is a recent report that anti-p185/HER2 antibody-mediated targeting of a cysteine proteinase inhibitor to a cathepsin B-containing intracellular compartment resulted in growth inhibition in two breast carcinoma cell lines including MCF-7 (41). Our model offers mechanistic insight into these observations, suggesting that growth impairment in these cells was related to defective ligand processing in the endosomes.

Collectively, these results identify the endocytic machinery as a critical component of growth factor receptor signaling which can be accessible and sensitive to specific proteinase inhibitors.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An anti-cancer compound for preventing tumor cell proliferation and/or inducing tumor cell apoptosis, which comprises a compound specifically targeted directly or indirectly at an endosomal enzyme involved in cellular processing of a growth factor, regulation of growth factor mediated signaling growth factor receptor turnover and tumorigenicity.

2. The anti-cancer compound as claimed in claim 1 wherein said compound is selected from the group consisting of chemical compounds comprising E-64, CA-074 and analogues thereof and antisense of cathepsins B, H, L, and S.

3. The anti-cancer compound as claimed in claim 1 wherein said growth factor is selected from the group consisting of IGF-I, IGF-II, TGFα, PDGF, EGF, HGFs, FGF, VEGF and others acting via tyrosine kinase receptors.

4. The anti-cancer compound as claimed in claim 1 wherein said compound is an antisense.

5. The anti-cancer compound as claimed in claim 4 wherein said antisense comprises a mRNA sequence capable of hybridizing to a cathepsin mRNA selected form the group consisting of sequence complementary to a cathepsin mRNA and fragments thereof.

6. The anti-cancer compound as claimed in claim 5 wherein said antisense comprises a sequence selected from the group consisting of a 300 bp fragment spanning nucleotides 511-810 of mouse cathepsin L gene set forth in SEQ ID NO:1 or functional equivalent fragment thereof of homologue cathepsin L gene.

7. A method for the treatment of cancer in a patient, which comprises administering to said patient a therapeutically effective amount of an agent for inhibition of an endosomal proteinase expression, whereby inhibition of a proteinase expression causes inhibition of growth factor degradation and cancer cell death.

8. The method as claimed in claim 7 wherein said proteinase is selected from the group consisting of endosomal cathepsins such as cathepsin B, H, L, and S.

9. The method as claimed in claim 7, wherein said cancer cells are metastases.

10. A method of screening compounds with anti-cancer activity, which comprises the steps of:
a) treating a cell line dependent on a growth factor receptor where a cathepsin is involved in its turn over with a compound; and
b) determining viability of the cell line, wherein apoptosis of said cell line is indicative of a compound having anti-cancer activity.

11. The method as claimed in claim 10, wherein said anti-cancer activity is an anti-metastatic activity.

12. The method as claimed in claim 10, wherein said growth factor receptor is selected from the group consisting of IGF-I-receptor, TGFα-receptor, PDGF-receptor, EGF-receptor, HGFs-receptors, FGF-receptor and VEGF-receptor.

13. The method as claimed in claim 10, wherein said cathepsin is selected from the group consisting of cathepsin B, H, L and S.

14. Use of a cell line for screening compounds with anti-cancer activity, wherein said cell line is dependent on a growth factor receptor where a cathepsin is involved in its turn over.

15. The use of claim 14, wherein said cell line is tumor H-59.

16. The use of claim 14, wherein said cathepsin is selected from the group consisting of cathepsin B, H, L and S.

17. The use of claim 14, wherein said anti-cancer activity is an anti-metastatic activity.

18. An anti-cancer compound, which comprises a compound blocking intracellular growth factor degradation whereby growth factor-induced cellular proliferation is inhibited.

19. The compound as claimed in claim 18 wherein said compound is selected from the group consisting of consisting E-64, CA-074 and chemical and functional analogues thereof, and antisense of cathepsins B, H, L, and S.

20. The compound as claimed in claim 18 wherein said growth factor is selected from the group consisting of IGF-I, IGF-II, TGFα, PDGF, EGF, HGFs, FGF and VEGF.
